(19) United States
(12) Patent Application Publication
(10) Pub. No.: US 2011/0171077 A1
(43) Pub. Date: Jul. 14, 2011

(54) VAPOUR DELIVERING DEVICE

(75) Inventors: Melodie A. McKenzie-Jones, Walsall (GB); Ron Jones, Walsall (GB); David W. Paddock, Staffordshire (GB); Jason Paddock, West Midlands (GB); Christopher J.N. Clews, West Midlands (GB)

(73) Assignee: Vapalight Limited, Birmingham (GB)

(21) Appl. No.: 12/514,192

(22) PCT Filed: Nov. 12, 2007
(86) PCT No.: PCT/GB2007/004304

§ 371 Date: Nov. 15, 2010

Related U.S. Application Data
(63) Continuation-in-part of application No. 11/686,687, filed on Mar. 15, 2007, now abandoned.

(30) Foreign Application Priority Data
Nov. 10, 2006 (GB) 0622418.2
Nov. 10, 2006 (GB) GB 0622418.2

(51) Int. Cl. A61L 9/03

(52) U.S. Cl. 422/125

(57) ABSTRACT

An apparatus is disclosed for delivering vaporised chemicals into the atmosphere. In one embodiment the device comprises a light bulb with a glass casing and a region of sinter glass. Alternative embodiments are disclosed in which the device comprises a retainer adapted to retain chemical carrying elements and fixing means adapted to secure the element to the heat source including low energy light bulbs. The chemical carrying element is preferably a sintered ceramic. The sintered ceramic preferably has a homogeneous particle and pore distribution and may be nanoporous.

This invention relates to an improved vapour-delivering device, especially suited to the evaporation of oils, scents, volatile chemicals and the like into the atmosphere.

It is well known to provide vapour delivering devices around the home or work environment. The devices deliver a vapour which is preferably fragranced to the surrounding air which may help in masking unwanted odours. Some fragrances have also been shown to help induce a feeling of well being to anyone inhaling the fragrance. Such devices may also be used to deliver non-fragranced vapours such as insecticides.

Traditionally, air fresheners have been in the form of stand-alone devices containing fragrant oil or the like. They have the advantage that they can be placed anywhere around the home or work, such as on a desk or windowsill. This can, however, be inconvenient and not all designs are pleasing to the eye.

An alternative, improved design, comprises a device which includes an electric heating element that can be plugged into a wall socket in a room. The electric heating element receives current from the supply to the socket which warms it up, in turn warming a pot of fragranced oil. Switching the device on or off allows the user to control the rate of evaporation of the oil and hence the fragrance. A disadvantage of such devices is that sockets around the home are often in inconvenient positions, for example behind a sofa or television, or may all be in use with other appliances.

It is also known to utilise vapour delivering devices to vapourise insect deterrents or insecticides.

It is an object of the present invention to provide an alternative vapour-delivering device.

According to a first aspect of the invention there is provided a vapour delivering device for the evaporation of a volatile chemical into the atmosphere by thermal diffusion comprising a low energy light bulb having a fitting suitable for connection to a light socket, a light emitting means and a casing surrounding the light emitting means, the casing being at least partially transparent to permit light to pass through the casing, emission means adapted to hold and allow the volatile chemical to evaporate by thermal diffusion, and a retention means adapted and arranged to connect to the light bulb and retain the emission means relative to the casing.

Preferably the emission means comprises a ceramic material.

Preferably a reservoir means is also provided in fluid connection with the emission means and providing a reservoir of the volatile chemical. This has the advantage of prolonging a burn off period of the device. Advantageously, it may be possible to arrange the reservoir such that the reservoir can be replaced, so enabling the supply of volatile chemical to be replenished without replacing the entire device.

Alternatively the emission means may be removed from the light bulb and replaced with a charged emission means or recharged by suitable means.

Preferably the light bulb is a low energy light bulb of the fluorescent type. The wattage is generally between 5 w and 40 w but this should not be viewed as limiting. The light may be delivered by fluorescence of gases within the casing. These kinds of light bulbs do not generate much heat, much less than arises from incandescent bulbs. An advantage of the invention is that the emission means is separated from the fluorescent tube or tubes of the light bulb. It has been found that the location of conventional vapour delivery devices in contact with the tubes causes a heat sink and the gases within the casing do not fluoresce correctly.

The light bulb may be of the spiral tube type. The retention means may encircle at least a part of the emission means and may be provided with means of engagement with at least a portion of the light bulb. Preferably the portion of the light bulb is remote from the fitting suitable for connection to a light socket.

The retention means may be provided as part of the emission means, or the reservoir and most preferably as part of the reservoir. The retention means may comprise walls of an elastic material adapted to retain the reservoir and emission means in place by elastic deformation of the walls. In a preferred embodiment the material is an elastomeric silicone material.

Alternatively the light bulb may be of the type provided with an aesthetically pleasing envelope enclosing a fluorescent type tube. The retention means, emission means and reservoir may be of any suitable type. In addition the envelope is provided with diffusion means. The envelope may be of a material allowing the diffusion of volatile chemicals through the envelope or may be provided with a number of apertures or orifices allowing the passage of chemicals to the atmosphere.

As has been described, the device in accordance with the first aspect of the invention is particularly suited to use with low energy fluorescent light bulbs used in place of traditional incandescent bulbs. An alternative aspect of the invention may be used with incandescent light bulbs of the more traditional type.

Pyrethrum has been found to particularly suited to the device and to be efficacious in the elimination of mosquitoes. The device, particularly but not exclusively, in accordance with the first aspect of the invention may be well suited to the delivery of chemicals used in the control of pests and diseases in horticulture, especially in intensive horticulture in greenhouses or poly-tunnels. The device may be readily, adapted to be used with larger low energy light bulbs now commonly used in greenhouses or poly-tunnels.

According to a second aspect the invention provides a vapour delivering device for the evaporation of chemicals into the atmosphere by thermal diffusion comprising a light bulb having a fitting suitable for connection to a light socket, a light emitting element and a casing which is at least partially transparent that surrounds the element to permit light to pass through the partially transparent portion, in which the casing is of glass and includes at least one integrally formed region of ceramic material which is adapted to hold a volatile substance whereby in use the light emitting element emits heat which heats the volatile substance causing at least some of it to evaporate.

Providing a device in the form of a bulb reduces the need for either a separate device or a free wall socket.

The volatile substance may comprise an oil based substance, which may be scented to release fragrance into the atmosphere. For example it may contain, menthol, fruit extracts, vanilla, lavender, or include other essential oils.

Alternatively, or additionally, the volatile substance may be a deodorising substance, an insecticide, a bactericidal preparation, a fungicide or some other chemical substance. It may be a therapeutic preparation such as for the treatment of asthma.

Providing a chemical holding layer of ceramic material which is integral with the glass casing of the bulb provides a device which looks very similar to an ordinary bulb as well as being robust and simple to manufacture.

The light bulb may have an incandescent light emitting element and may have either a bayonet type or screw cap type fitting. This allows the device of the invention to be used in place of a conventional light bulb in a lamp or pendant fitting, a wall light or ceiling light or the like. It will be understood that this list is not intended to be limiting.

In one embodiment the ceramic material used has been sintered glass. This has been found to be particularly advantageous with incandescent light bulbs. Other ceramic materials, particularly sintered ceramic materials may be used.

The sintered glass layer may be fused or bonded to the glass casing. It is preferably fused by being applied to the glass casing when the bulb is formed, perhaps before the molten glass used to form the casing has fully cooled. However it is applied, it is preferred that the sintered glass forms an integral part of the casing. The sintered glass layer is preferably at least partially transparent.

The sintered glass layer may alternatively be fixed to the glass bulb by a coupling which is itself secured to the bulb. The coupling may comprise a pin which may pass through the glass casing. The pin may carry a thread which protrudes from the glass casing and which co-operates with a complimentary thread on the sintered glass layer. This arrangement may be advantageous as it allows the sintered layer to be detached from the glass casing.

The thread may be a universal twist lock fastening. In this arrangement the glass layer may be pre-shaped to compliment the shape of the glass casing onto which it is to be fitted.

The sintered glass layer may be applied around a portion of the bulb furthest from the fitting. In this way the sintered glass will be at the top of the bulb when it is fitted to most standard table or desk or floor standing lamps. This allows the fragrance to evaporate straight upwards as the bulb warms up, being circulated around a surrounding area by convection.

In one arrangement, a pin of thermally conductive material may be provided within the layer which extends from inside of the bulb casing outwards through the sintered layer. Alternatively, it may extend from a point in or touching the casing through the sintered layer. The pin may help carry heat through the layer to ensure optimum temperatures in the layer for controlled evaporation.

The pin may be metal, for example molybdenum or Nickel/Iron alloy. Alternatively it may be made of glass, glass fibre, plastics or polymer such as PTFE.

More than one pin may be provided, dependent upon the required thermal characteristics and other features of the bulb.

This pin may be integrally formed with the sintered glass layer, preferably fused in place during manufacture. Alternatively, it may be releasable from the sintered glass later and therefore function as the coupling described hereinbefore. It may be provided with, for example, a universal twist lock.

The pin may be an integral part of the lamp formed during manufacture or may be bonded onto the lamp after manufacture.

The sintered layer may have a uniform thickness or may have a greater thickness closer to the pin than in regions farther from the pin.

The sintered layer is able to absorb a volatile oil. The oil may be supplied with the bulb or may be supplied separately. It is envisaged that a wide range of oils may be supplied which can be purchased separately.

The device may be provided in the form of a range of different shapes and sizes allowing different devices to be used to replace conventional bulbs. For example, the bulb may be a 40 watt, 60 watt, 100 watt or 150 watt bulb as is conventional for domestic lighting applications.

The sintered glass layer and/or the glass casing may be coloured. In the arrangement where the layer can be removed, a different effect can then be obtained by attaching layers of different colours or opacities. It is therefore envisaged that a wide range of different sintered layers may be provided.

The layer may be formed into a shroud which substantially surrounds the whole of the casing of the bulb.

Thus, according to a third aspect the invention provides in combination a fragrance-emitting device according to the first or second aspect and fragranced oil.

According to a fourth aspect the invention provides a ceramic carrier and emission means for volatile chemicals so constructed and arranged as to comprise a layer conforming to the shape of at least part of a light bulb, the layer including a coupling for attachment of the layer to the bulb.

The coupling may comprise an opening having an internal thread suitable for engagement with a corresponding male thread protruding from the bulb. The opening may be formed by a nut which is held captive within the ceramic material, perhaps fused in place.

The ceramic layer may be coloured and may be impregnated with a volatile chemical substance such as an oil.

According to a fifth aspect the invention provides a light bulb having a fitting suitable for connection to a light socket, a light emitting element and a casing which is at least partially transparent that surrounds the element to permit light to pass through the partially transparent portion, in which the casing includes a connector which is adapted to co-operate with a carrier of ceramic material which contains a volatile chemical.

The connector may comprise a pin which extends from the casing of the bulb outwards. It may carry a male thread which is adapted to co-operate with a complimentary thread on a carrier. The thread may comprise a universal twist lock.

According to a sixth aspect the invention provides a vapour delivering device for the evaporation of chemicals into the atmosphere by thermal diffusion comprising an emission means and a connection means adapted to secure the emission means to or in the vicinity of a thermal source, wherein the emission means comprises a ceramic material.

Preferably the emission means comprises a substantially homogeneous ceramic material.

The connection means may comprise a retaining means adapted to retain the emission means and a fixing means adapted and arranged to secure the emission means in place, adjacent to and spaced from the thermal source. The thermal source may be a light bulb or other source of heat.

Preferably the chemical carrying element comprises a disc of ceramic material. In one embodiment the disc is formed of sintered glass. Other suitable sintered ceramics may be used.

In an alternative embodiment the chemical carrying element comprises a elongate member of ceramic material. The elongate member may be in the form of a cylindrical or cuboid rod. Alternatively an elongate sheet of ceramic material may be utilised. It is also envisaged that the elongate member may be formed in a curved or spiral form to suit the light bulb to which the element is to be affixed. The choice of shape of the chemical carrying element may be made on aesthetic decisions

The ceramic may be formed by pressing, moulding or extrusion. Pressing may be more suited to ceramics formed into discs or some rods. Alternatively, the ceramic may be formed by extrusion. This may be used to form rods and cylinders. More complex shapes may be formed by moulding of the ceramic. The retainer may be formed of a silicon material or a suitable plastics material which may enclose the chemical carrying element. Suitable orifices may be provided in the material or the material may be selected so as to allow diffusion of the chemical through the material under conditions where diffusion is desired.

Not all ceramic materials have been found to efficacious and to provide consistent and reliable results. Preferably the ceramic material has an overall porosity by volume in the range 10% to 80%. Particularly referred ranges of porosity may be 30% to 50%. The most desirable porosity range may vary with both the volatile chemical and chemical carrier to be evaporated, and the desired burn off period. A longer burn off may be achieved by using a ceramic material with a higher percentage porosity by volume.

Suitable ceramic materials may comprise sintered ceramics such as high silica borosilicate glasses or soda glasses. Suitable glasses may be Pyrex, Duran or alternative sintered ceramics. Preferably the ceramics are heat treated or annealed such that the ceramic grains have rounded angles.

Preferably the ceramic material comprises ceramic particles having a relatively narrow particle distribution size. In a preferred ceramic the majority of finer and coarser particles have been removed leaving a substantially mono size particle distribution. One particularly suitable distribution is a particle size from 40-80 microns. In other suitable ceramic materials the average particle size is 200 to 300 microns.

Preferably the ceramic particles are rounded. In some embodiments this is achieved by annealing or sintering the ceramic. The skilled man may substitute alternative methods of obtaining suitably rounded grains in the ceramic.

It has also been determined that a substantially mono-size pore size distribution is also preferable. A pore size range between 10 and 100 microns is advantageous. One particularly suitable pore size range has been found to be 10-20 microns. However, a pore size range between 0.2 microns and 110 microns may be utilised.

The retaining means may comprise a disc holder formed of plastics material. The holder may comprise a first portion adapted to contain the disc of ceramic material and a second portion adapted and arranged to locate over, and secure to the first portion so retaining the disc. The first and second portions may each be provided with one or more apertures.

Preferably the second portion is provided with at least one aperture.

The first and second portions are preferably formed of a thermo-resistant plastic.

The fixing means may comprise a metal pin. The metal pin may be integrally formed with the heat source or may be arranged to engage with the heat source. Alternatively the fixing means may be an adapter with a suitable adhesive between the adapter and the heat source. In another embodiment the retainer is fixed to the casing by means of a suitably shaped suction cup. The retainer may be used with incandescent or low energy light bulbs.

In one embodiment the heat source is a conventional electrical bulb. The bulb may be a conventional shape or maybe a candle bulb or even a long life compact fluorescent bulb.

It is also envisaged that the heat source may be a camping gas light or other source of heat.

In an embodiment in which the heat source is a camping gas light, a conventional globe of the gas lamp or a conventional metal cap located above the globe may be replaced with a globe or cap comprising at least partly of the chemical carrying element adapted to hold a volatile substance whereby in use heat from the camping gas light heats the volatile substance causing at least some of it to evaporate.

The chemical carrying element may comprise a disk retained within a metal cap for a camping gas light. Other shaped inserts are also envisaged.

Alternatively an insert may be provided in the globe surrounding the gas light.

Retaining means may be provided which may allow the disc to be removed and replaced or may allow recharging of the chemical carrying element with the volatile substance.

Fixing means may be provided. These may secure the retainer, combined globe and retainer, or combined cap and retainer, to the thermal source, in this case the camping gas light unit.

In another embodiment, suitable retaining means and fixing means are provided to secure the chemical carrying element to or adjacent a fluorescent tube as the thermal source.

According to a seventh aspect the invention provides an emission means for use in a vapour delivering device according to any previous aspect of the invention.

According to an eighth aspect the invention provides a retaining means suitable for retaining a chemical carrying element and adapted to releasably engage with fixing means provided on or fixed to a thermal heat source.

According to an ninth aspect the invention provides a volatile substance suitable for use in a vapour delivering device according to the first, second and sixth aspects of the invention.

According to a tenth aspect the invention provides a vapour delivery device for the evaporation of chemicals into the atmosphere by thermal diffusion comprising a chemical carrying element; connecting means connecting the chemical carrying element to a thermal source wherein the chemical carrying element comprises a ceramic material.

According to an eleventh aspect the invention the provides a vapour delivery device for the evaporation of a volatile chemical into the atmosphere comprising a thermal source and an emission means provided in a spaced relationship to and thermally linked to the heat source wherein the chemical carrying element comprises a ceramic material.

According to a twelfth aspect the invention the invention provides a kit of parts comprising at least one of:

an emission means;
a retaining means;
a reservoir suitable for connection to an emission means, and
a volatile chemical suitable for use in a vapour delivery device the parts when assembled forming a vapour delivery device according to a first, fourth, fifth, sixth, tenth or eleventh aspect of the invention.

There will now be described, by way of example only, embodiments of the present invention with reference to the accompanying drawings of which:

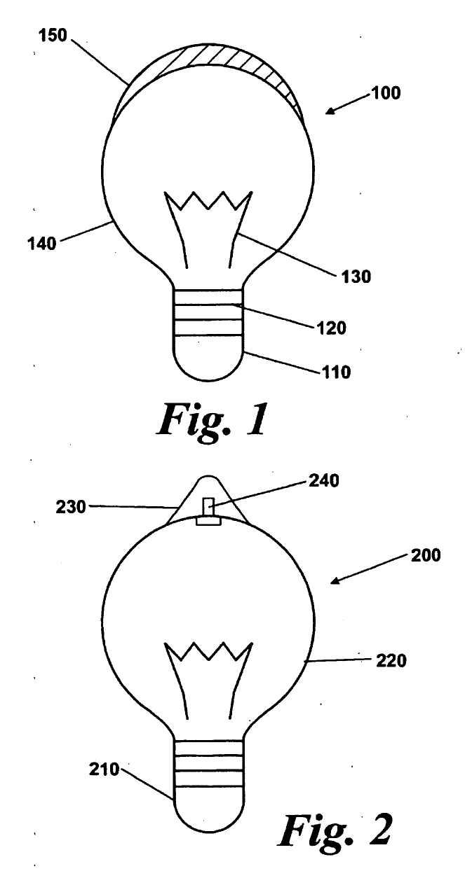

FIG. 1 is an illustration of a first vapour delivering device in accordance with the second aspect of the present invention;

FIG. 2 is an illustration of a second vapour delivering device in accordance with the second aspect of the present invention;

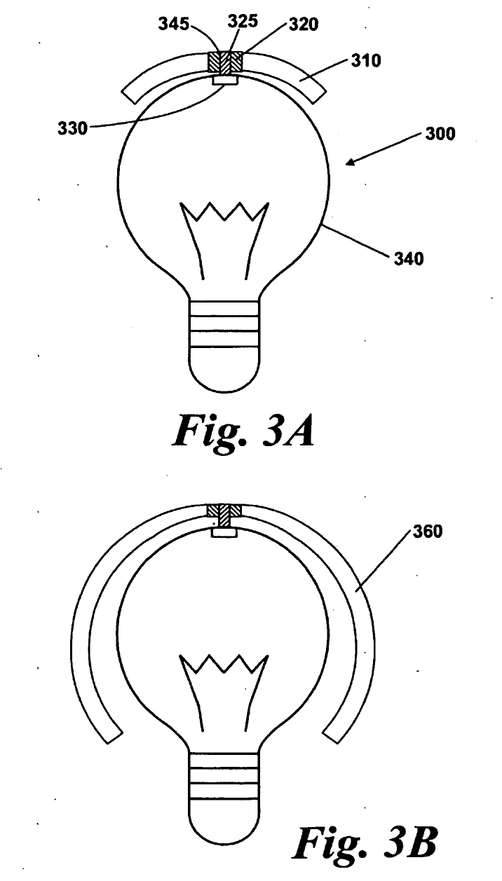

FIG. 3a is an illustration of a third alternative vapour delivering device in accordance with the second aspect of the invention;

FIG. 3b is an illustration of a fourth alternative vapour delivering device in accordance with the second aspect of the invention;

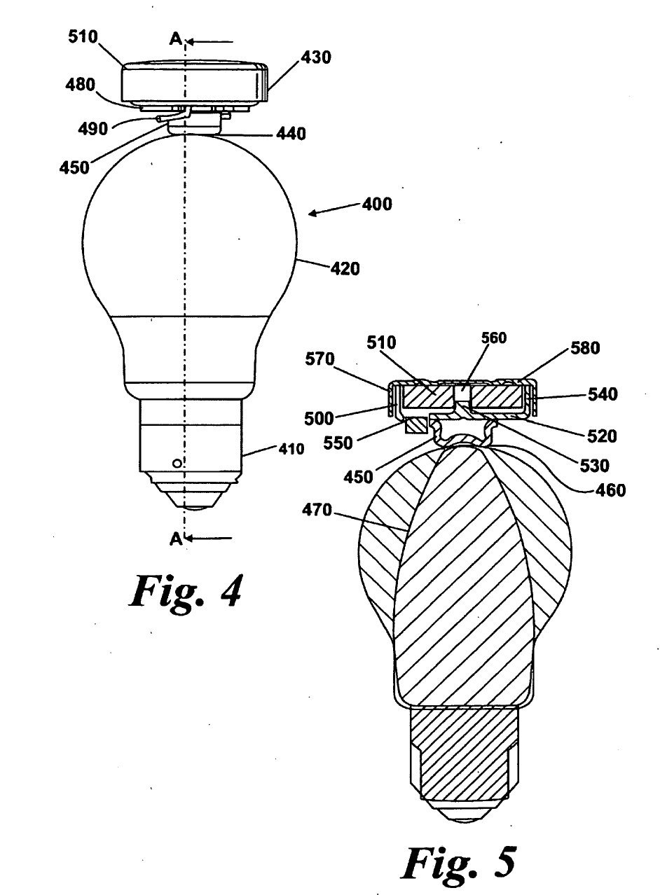

FIG. 4 is an illustration of a fifth vapour delivering device in accordance with the sixth aspect of the invention;

FIG. 5 is a cross-section along the line A-A of FIG. 4; and

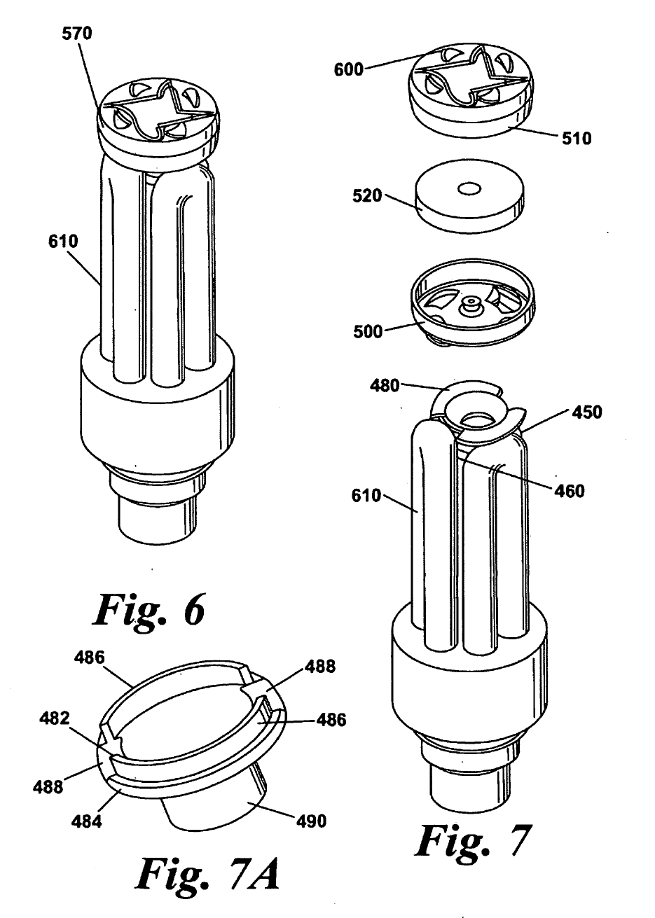

FIG. 6 is an illustration of a sixth vapour delivering device in accordance with the sixth aspect of the invention;

FIG. 7 is an exploded view of the device of FIG. 6;

FIG. 7A is a perspective view of an alternative fixing means;

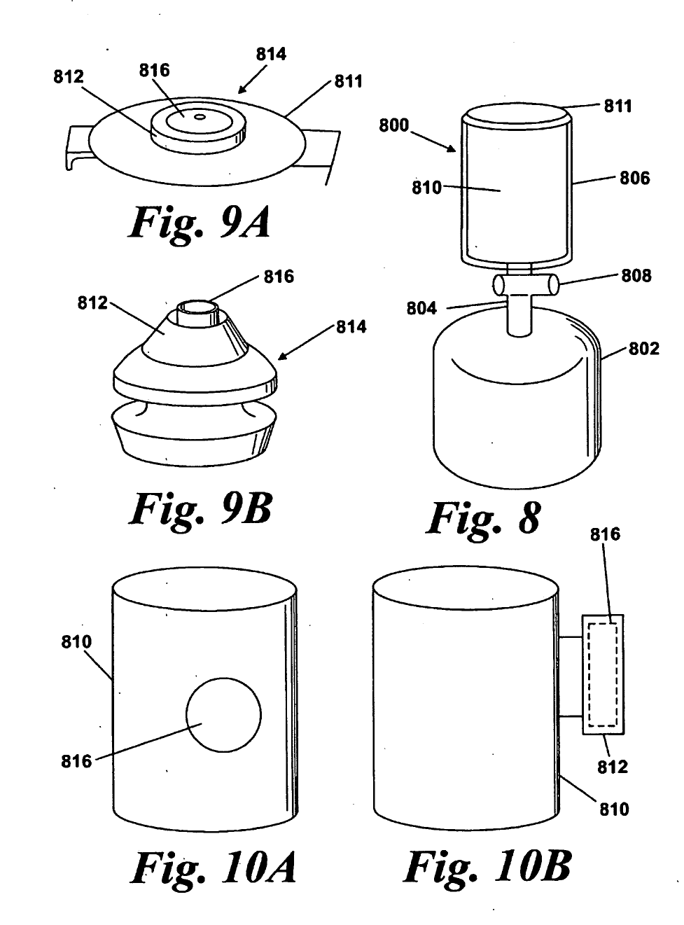

FIG. 8 is a diagrammatic illustration of a conventional camping gas lantern;

FIGS. 9a and 9b are illustrations of a cap for a lantern incorporating a vapour delivering device;

FIGS. 10 and 10a are illustrations of a globe for a lantern incorporating a vapour delivering device;

FIGS. 11a to 11d are perspective views of a low energy fluorescent bulb and a vapour delivering device with alternative reservoirs attached to the fixing means in accordance with the invention;

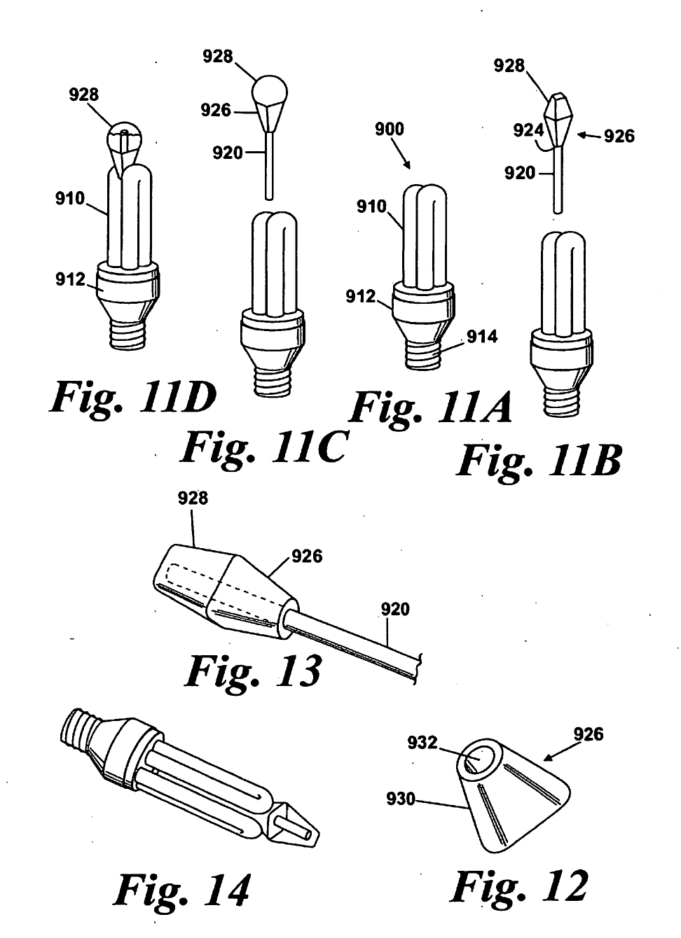

FIG. 12 is a more detailed view of the fixing means;

FIG. 13 is a detailed view of the fixing means attached to a ceramic rod and a reservoir;

FIG. 14 is an alternative view of the device in position on the bulb;

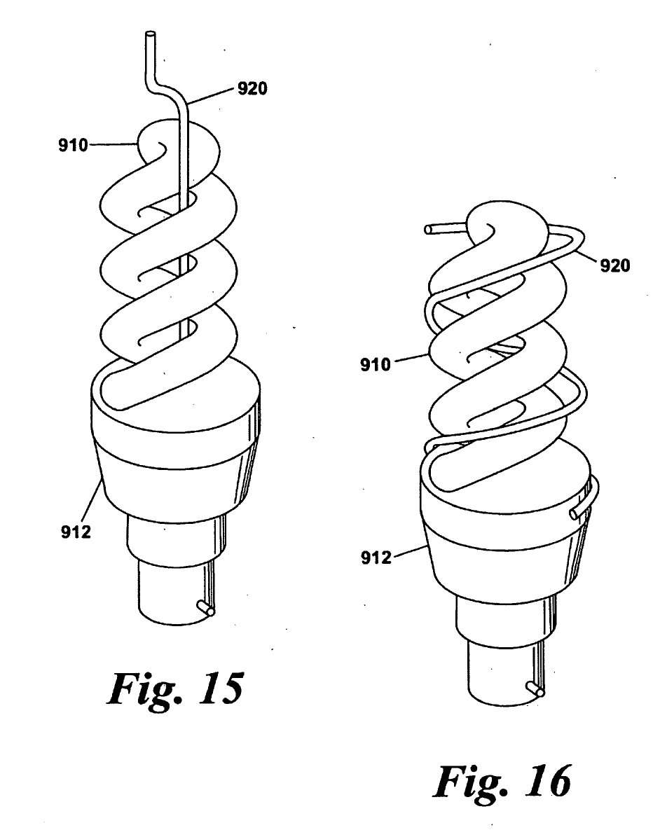

FIGS. 15 and 16 illustrate alternative emission means suitable for use with low energy light bulbs incorporating a spiral tube; and

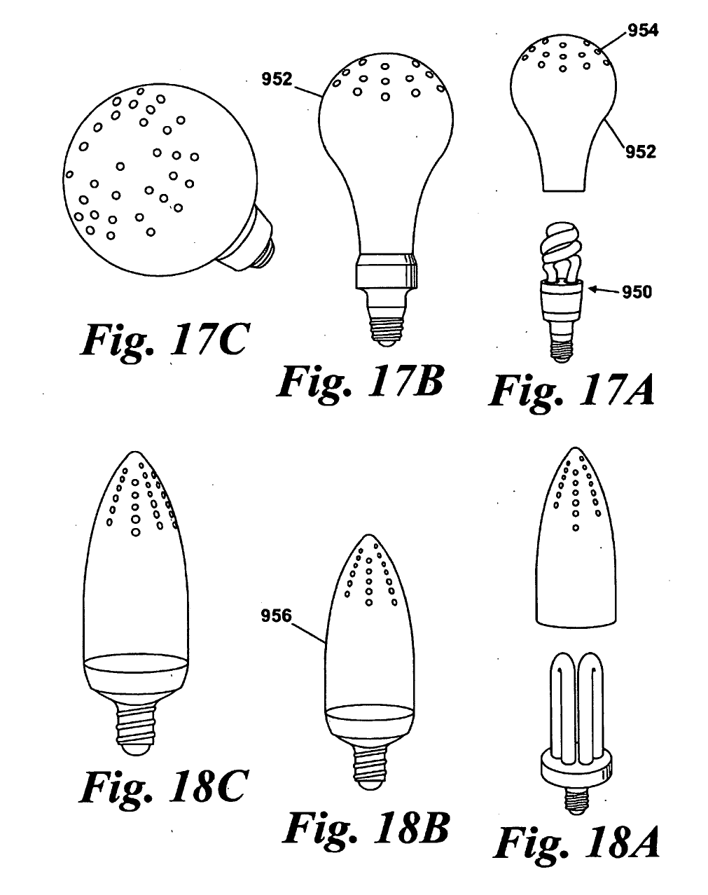

FIGS. 17 and 18 are views of a detachable envelope for use with a vapour delivering device in association with a low energy light bulb.

FIG. 1 shows a vapour delivering device 100 which functions as a light bulb. As such it can be used to replace any conventional light bulb around a home or workplace. It comprises a conductive base 110 having a screw thread 120. The base and thread are shaped to co-operate with a light fitting (not shown) having a complimentary thread.

Attached to the base 110 is a light emitting element 130 which in the example shown is a resistive filament. The filament draws current from the supply to the light socket and as current passes through the filament it heats up to such an extent that it emits light. Such technology is well known to the man skilled in the art.

The filament is protected by a glass casing 140 and a suitable gas is sealed within the casing 140 to ensure that the filament operates effectively. The glass casing 140 in the example is totally transparent although it may be opaque. It is dome shaped but could be other shapes as desired without impairing the function of the device.

At the top of the glass casing 140 is a layer of ceramic material 150 which is integrally formed with the casing 140. The ceramic material will be further described below. However an advantageous form of ceramic material has been found to be sintered glass and the invention will be further described in relation to the use of sintered glass. It will be understood however that this is not limiting and as will be further described alternative materials may be used in place of sintered glass as will be understood by the man skilled in the art. This ceramic or sintered glass layer 150 is porous. A volatile fluid (not visible in the figures) is absorbed within the sintered glass layer 150. The fluid is selected to have properties that permit it to evaporate as it is heated by the element. In the example shown the fluid contains a fragrance and as it heats the ceramic material allows the fragrance to pass into the air around the bulb. The fluid may be selected depending on the pore size and the desired use, whether as an air freshener, insect repellent or other use. The fluid may be a volatile oil.

An alternative embodiment of a vapour delivering device 200 is shown in FIG. 2. This device is similar to that of FIG. 1 of the accompanying drawings having a base 210 and casing 220 but in this device a sintered layer 230 is provided which is thicker and is penetrated by a metallic pin 240 which passes through the glass casing. The pin 240 helps to carry heat from the inside of the casing into the sintered glass layer 230. It is envisaged that in at least some arrangements this may permit a thicker layer of sintered glass to be used whilst ensuring it heats through rapidly when the bulb is switched on. The pin 240 also helps secure the sintered glass layer.

A third alternative arrangement of a vapour delivering device 300 is shown in FIG. 3 of the accompanying drawings. In this arrangement the sintered glass portion comprises a removable carrier 310. It includes a captive nut 320 which has an internal female thread 325. A pin 330 is secured to the glass casing 340 of the device in a similar manner to the embodiment of FIG. 2 and carries an external male thread 345 which compliments the female thread of the nut. This arrangement allows the sintered glass carrier to be removed from the glass casing if desired.

FIG. 3b shows a modification to the arrangement of FIG. 3a in which the sintered glass portion comprises a shroud 360 which extends completely around the bulb casing. The shroud 360 is coloured to alter the light that is emitted into a room allowing it to be used to create different feelings of personal well being. Alternatively it may be used as a night light. The volatile liquid may be a decongestant if desired.

FIG. 4 shows an alternative embodiment of a vapour delivering device 400. The device comprises a conventional light bulb 40 having a base 410 and a casing 420. In this embodiment the light bulb is a thermal source of radiant heat as well as a source of light.

A retainer 430 is located an end of the light bulb remote from the base 410. The retainer 430 is secured to the casing 420 of the light bulb by suitable means, in this case adhesive 440 and an adapter 450 thus connecting the emission means to the light bulb. The adhesive should be suitable for use in situations reaching a high temperature and also able to sustain wide temperature fluctuations.

LOCTITE® 350 supplied by Henkel Technologies and RTV 382 supplied by Intek Adhesives have been found to be suitable adhesives.

The adapter 450 is shaped to abut with a conventional light bulb. A lower surface 460 of the adapter is shaped to be able to abuttingly engage the light bulb and adhesive is applied to the lower surface 460 to secure the adapter to the casing 420 of the light bulb.

Preferably the lower surface 460 of the adapter is shaped such that the same adapter can be used on more than one form of conventional light bulb—in FIG. 5 a candle light bulb 470 is also shown and as can be seen the lower surface 460 can abuttingly engage an end of this form of light bulb too.

The adapter 450 is also provided with lugs 480 arranged to cooperate and engage lugs on the retaining means. Other releasable engaging means could be used.

The retaining means shown in FIG. 7 comprises a first portion 500 arranged to contain a disc 510 of sintered glass. The first portion 500 comprises a base 520 having a central locating pin 530, side walls 540 and apertures 550 on the base 520. Portions of the base 520 may be pressed out in manufacture to form the apertures 550 and lugs 490 which engage with cooperating lugs 480 on the adapter.

In this embodiment the chemical carrying element comprises a disc 510 of sintered glass. The disc 510 has a central aperture 560 arranged to locate over the locating pin 530 of the first portion of the retaining means. Alternative shapes may be used.

The retaining means further comprises a second portion 570 arranged to securely but removably locate over the first portion 500. The second portion 570 comprises a cover 580, arranged in use to be parallel to the base 520, and side walls 590 extending in use from the cover 580 towards the first section 500. The cover 580 is provided with a number of apertures. These may be seen in FIGS. 6 and 7. The apertures 600 allow passage of the volatile substance from the carrying element, disc 510, into the atmosphere when heated.

The side walls 570 of the second portions are arranged to releasably engage with the side walls 540 of the first portion. They may for example be a push fit.

The first and second portions together with the adapter may be made of a suitable plastics material. A suitable plastics material should have good mechanical qualities, be chemically resistant to the chosen vapours and be heat resistant. Suitable plastics materials include Zytel® nylon, RYNITE® and CRASTIN® all supplied by DuPont. It will be understood that other plastics having suitable properties may be substituted, or other non-plastic materials having suitable properties could be used. Silicone plastics may also be suitable.

Turning to FIGS. 6 and 7 these shown a retaining means and adapter arranged to fix the vapour delivering device to an energy saving bulb of the compact fluorescent type. As can be seen the retaining means is very similar to that of FIGS. 4 and 5. The adapter 450 is arranged such that the lower surface 460 is able to fit within the tubes 610 of the light bulb.

Alternative adaptors could be utilised. Further variations are envisaged in order to fix the retaining means to other types of light bulb.

An alternative adaptor 482 is illustrated in FIG. 7A. The adaptor 482 comprises an annular base 484 having first and second upstanding walls 486 extending around the periphery of the base 484. The first and second upstanding walls are separated by a lip 488 on either side of the base. The base and walls are sized so that a retainer may be mounted within the walls or the base and walls may be substituted for a base of a retaining means.

A depending cup 490 is provided below the annular base 484. The cup is arranged so as to provide a means of mounting the adaptor on a light bulb. Preferably the cup is mounted by means of a suction fit on the light bulb enabling the adaptor to be used on upwardly or downwardly directed light bulbs. The exact size of the cup 490 of the adaptor may be varied depending on the shape of the light bulb to which the adaptor is to be fitted. In a variation particularly suitable for use with light bulbs of the low energy variety the cup may be sized to fit between the fluorescent tubes and may be made of a resilient material enabling a firm push fit. The exact material and sizing would be readily apparent to a man skilled in then art.

It has been found that it is desirable to connect the emission means to the light bulb such that the emission means is adjacent to and spaced from the tubes of a fluorescent tube low energy bulb so that the emission means does not act as a heat sink and prevent the correct operation of the fluorescence of the gases in the tube. Preferably the connection means connecting the emission means to the light bulb is formed from a heat resistant non heat conductive material such as a plastics or silicone material.

Turning now to FIGS. 8 to 10, FIG. 8 shows a conventional camping gas lamp 800 having a source of gas 802, a pipe 804 delivering gas to a lamp portion 806. A means of controlling delivery of the gas is provided by a valve 806.

The lamp portion 804 comprises a globe 810 conveniently formed of glass, either opaque or clear, and a cap 811. This cap 811 is conventionally made of metal.

Means, not shown, are provided for securing the cap in place over the globe 808. Commonly hanging means are also provided whereby the lamp can be suspended.

FIG. 9a shows an embodiment of the invention in which a retaining means 812 is provided secured to the cap 811. The retaining means may be similar or identical to that used for the light bulbs or an alternative form may be used. A fixing means may be provided to fix the retainer 812 to the cap 810. The fixing means may comprise an adapter and adhesive as before or may be different.

Alternatively, retaining means may be formed integrally in the metal cap. The retainer may contain a chemical carrying element 814 in the form of sintered glass in a disc 816.

A further alternative comprises providing retaining means 812 and fixing means suitable for affixing to a portion of the globe 810, as generally indicated in FIG. 10b. It may be desirable to use a glass retaining means.

The chemical carrying element may be formed integrally with the globe as generally indicated in FIG. 10a. Alternatively the retaining means may be integral with the globe 810 and the disc 816 may be removable.

A man skilled in the art may adapt variations in the fixing and/or retaining means without departing from the invention.

A number of ceramic materials have been investigated for their suitability as the chemical carrying means. Of these a number of sintered ceramics have been found to be particularly effective. Coralith™ C5 available from Fairey Filtration Systems and Alumina A14 from Ceram Tec have been found to be a suitable ceramics together with traditional sintered glass and to provide suitable retention of volatile substances.

These ceramics are alumina glasses or borosilicate glass ceramics. Suitable examples are sintered glasses used in filtering and glasses such as Pyrex™ and Duran™. Other materials may also be suitable. Micro or nanoporous materials may have suitable properties.

Investigation and experimentation has shown that a suitable ceramic must have a number of characteristics. In particular it has been found that the ceramic should have a grain size that is substantially homogeneous and that finer and coarser grains should be removed from the ceramic material. A grain size in the region of 20 to 400 microns has been used. One suitable ceramic has a grain size in the region of 40 to 80 microns while other suitable ceramics have a grain size between 200 and 400 microns and 200 to 300 microns in particular. Particles between 0.1 and 100 μm may be used depending on the properties of the volatile substance.

Preferably both larger and finer sinters are not incorporated. It is believed that a homogeneous distribution of particle size may facilitate the retention of volatile substances in use and so prolong the working life of a charged element.

It seems to be advantageous for the ceramic to have grains that have rounded edges. This may be achieved by sintering the ceramic as in sintered glass or by otherwise annealing the ceramic.

The ceramic should also have a relatively narrow range of pore diameters and preferred materials have pore diameters in the range of 0 to 100 microns. One particularly suitable material has been found to have pore diameters in the range of 10 to 20 microns while another suitable material has pore diameters in the range 20 to 30 microns.

In a form of the invention the ceramic material has pore diameters of 7-20 microns. The pore size may be much smaller for example 10-9 m to 10 3 m.

Overall porosity of the ceramic varies with the pore size distribution. It is desirable that the overall porosity is selected such that the burn off time of the volatile chemical is prolonged. If the burn off is too rapid then the volatile chemical is dispersed too quickly. The burn off period increases with the porosity so in general a higher porosity ceramic will increase the burn off period. The exact preferred porosity may vary with the volatile chemical and carrier oil utilised. Porosity values from 10% to 80% have been used depending on the chemical and carrier oil. In general though a porosity in the region of 20 to 50% has been found to provide suitable burn off periods and a particularly desired range is 30 to 40% porosity.

Sintered glass having an overall porosity of around 35% by volume may be formed from a thermal, shock-resistant borosilicate glass such as Pyrex or Duran.

It is envisaged that the pore size of the chemical carrying element may be varied depending on surface tension and wetting properties of the volatile substance. The pore size may be less than 0.1 microns or greater than 100 microns. Nano-porous materials may also be particularly suitable for use as the chemical carrying element.

Preferably the material of the chemical carrying element is non reactive with the volatile substance.

The applicant has realised that a vapour delivering device may be provided for evaporation of chemicals into the atmosphere by thermal diffusion comprising a chemical carrying element in combination with (secured to or integrated with) a thermal heat source, the chemical carrying element being adapted to release a chemical over a period of time under the influence of heat from the thermal heat source.

Alternatively a vapour delivering device may be provided for evaporation of chemicals into the atmosphere by thermal diffusion comprising a chemical carrying element and fixing means adapted to fix the chemical carrying means on or in the vicinity of a thermal heat source, the chemical carrying element being adapted to release a chemical over a period of time under the influence of heat from the thermal heat source.

The thermal heat source may be a hand drier. The chemical carrying element may be secured to the hand drier or in the vicinity of the hand drier.

Preferably the chemical carrying element is one of ceramic material, a sintered glass, a micro porous material or a nanoporous material.

Alternative embodiments of the invention will now be described. FIG. 11a shows a typical low energy light bulb 900 of the kind having two curved fluorescent tubes 910 which protrude from a base 912 of the light bulb in an opposite direction from a fitting 914 suitable for connection to a light socket. The fitting 914 may be of any conventional type, for example a screw fitting or a bayonet fitting.

FIG. 11b illustrates a device in accordance with a first aspect of the invention comprising a ceramic rod 920 sized to fit between the fluorescent tubes 910 of the light bulb. The rod comprises chemical emission means and is formed of a suitable ceramic. The rod is also adapted to hold or retain a quantity of chemical before emission occurs as a result of thermal diffusion. The rod has a first end 922 and a second end 924 remote from the first end. A retaining means 926, which will be further described below is mounted on the second end 924 of the rod 920. The retaining means has mounted thereupon and opposing the ceramic rod a reservoir 928 adapted to hold a volume of volatile chemical and arranged to communicate with the ceramic rod to replenish the volatile chemical in the ceramic rod.

As can be seen in FIG. 11c the shape of the reservoir may be varied without affecting the performance of the device. In use the ceramic rod is inserted between the tubes 910 of the light bulb. The retaining means is arranged to hold the ceramic rod in position in the light bulb. The retaining means may comprise a resilient silicon material arranged in a suitable shape to retain the ceramic rod in the desired location between the tubes.

It has been found that spacing of the ceramic rod away from the fluorescent tubes of the light bulb is important to the functioning of the device. In incandescent bulbs the ceramic may be in contact with the envelope of the bulb or integral with the envelope. However, it has been found that if the ceramic rod is substantially in contact with the tubes of the low energy light bulb the ceramic rod acts a heat sink and the tubes of the light bulb do not fluoresce correctly. It is therefore important that the retaining means position the emission means close to but not in contact with a substantial part of the tubes. The emission means is spaced from but in thermal connection with the light emitting means.

A more detailed view of the retaining means 926 is shown in FIG. 12. The means 926 comprises a rounded three sided pyramid 930 formed of silicone material. The means 926 may be formed of any suitable material of which silicone is a single example. The material is preferably resilient allowing the retaining means to be secured in place by a push fit between the tubes of the fluorescent tubes of the light bulb. The material should also be heat resistant. A suitable material is ALSIL 18357 supplied by SRM Mouldings. Alternative materials would be obvious to a man skilled in the art. The pyramid 930 is provided with an orifice 932 adapted to securely receive and retain the ceramic rod 920.

FIGS. 13 and 14 show the retaining means and a reservoir 928 mounted thereon in more detail. As can be seen in more detail in FIG. 13 the reservoir 928 is mounted on the retaining means and may be integral therewith. Alternatively a secure connection means may be provided. In this example the ceramic rod 920 extends through the retaining means and into the reservoir 928. The fluid in the reservoir is thus in fluid communication with the ceramic rod and can replenish the supply of volatile chemical as it is emitted from the rod. It is envisaged that replacement reservoirs may be produced and sold and that the reservoir may be formed of a suitable material to contain the chemical and to connect to the rod. The reservoir may be provided with a seal that is broken when the rod is connected to the reservoir. As can be seen in FIG. 14 the rod and retaining means may be inserted between the tube and the reservoir projects from an end of the light bulb. This may be in the form of a thin membrane of silicone over an aperture sized to receive the rod.

The rod may contain a coloured indicator material that changes colour as the volatile chemical is emitted. This may advantageously indicate the amount of chemical remaining and endure that the reservoir may be replaced in sufficient time to maintain the emission of the volatile chemical at the desired level. A coloured indicator may be provided in other forms of the chemical carrying element or emission means.

The rod may be a cylindrical rod or may be a cuboid. In some embodiments the rod may be encased in a suitable material and provided with suitable means of permitting the volatile chemical to be emitted from the emission means. This may be in the form of apertures or orifices provided in the encasing material. Alternatively the material may be selected to have properties so as to permit diffusion through the encasing material. The emission means may be encased so as to prevent the volatile material diffusing before the emission means is placed in the light bulb in use. Alternatively the emission means may be encased to provide greater structural strength since these ceramics may be brittle if the rod or other shape is particularly thin. In a further alternative embodiment the emission means may be in the form of a thin sheet that may be inserted between the tubes of the light bulb in use. In the embodiments described so far the emission means is inserted between the tubes. In another embodiment the emission means may be retained in place externally of the tubes.

The ceramic material may be extruded, pressed or moulded and the latter in particular enables the material to be formed into more complex shapes. These may be utilised with other forms of light bulb such as those shown in FIGS. 15 and 16. In these the fluorescent tube 910 is formed into a spiral shape. The emission means 920 may be formed into a narrow curve that may be inserted within the spiral formation of the fluorescent tube and connected to the light bulb. Alternatively as illustrated in FIG. 16 the emission means 920 may be formed into a complex spiral that encircles the spiral form of the fluorescent tube 910.

Further alternative embodiments are illustrated in FIGS. 17 and 18. FIG. 17 a shows a compact fluorescent light bulb 950 having a detachable envelope 952 formed of a glass or a heat resistant plastics material or other suitable material. FIG. 17b shows the detachable envelope in position over the light bulb 950. It is evident that the fluorescent tubes of the light bulb are not visible to a user. In this embodiment the emission means used may be of any suitable and convenient form and need not be selected with a view to the aesthetic appearance of the emission means. The envelope is provided with a number of orifices 954 in a part of the envelope remote from the fitting for a light socket. The orifices 954 allow the diffusion of the volatile chemical from the emission means to the atmosphere. FIG. 18 illustrates a similar embodiment in which the envelope is of a candle shape. The candle shaped envelope is provided with orifices at the end of the envelope remote from the light fitting. These embodiments are particularly suitable for light bulbs which are to be used in a depending position.

It will be understood that the temperatures reached in or near a low energy light bulb are significantly lower than the temperatures reached in or near an incandescent bulb. The temperature near a low energy bulb may be less than 100 degrees C.

The invention has been described in relation to household incandescent and low energy light bulbs. The device may also be used in combination with the type of bulbs used in greenhouses or poly tunnels. These are much larger, typically 12″ to 18″ long fluorescent tubes. These bulbs may be used of lighting or may be utilised to provide a form of gentle heat. The device may be used with these light bulbs to provide a supply of a volatile chemical that is used to treat or prevent plant diseases. The device may be used in combination with light bulbs provided with a black out cover such that they provide only heat and not light. The emission means may be much larger than those used in combination with the smaller light bulbs.

It is envisaged that the device may be adapted for use with LED light sources.

The device has been found to be particularly effective in providing a supply of Pyrethrum as a gas in the atmosphere. Pyrethrum is used to kill mosquitoes. It has been found that the chemical is effectively diffused by the device and that desirable and consistent kill rates are achieved. It is believed that this is the first time that this chemical has been diffused in this way. Attempts to thermally diffuse Pyrethrum have been made in the past but have not been successful.

1.-55. (canceled)

56. A vapor delivering device for the evaporation of a volatile chemical into the atmosphere by thermal diffusion, the vapor delivering device comprising:
(a) a low energy fluorescent light bulb having a fitting suitable for connection to a light socket, said light bulb comprising:
(i) a light emitting element; and
(ii) a casing surrounding the light emitting element, said casing having a portion that is at least partially transparent to permit light to pass through said partially transparent portion;
(b) emission means for holding said volatile chemical and allowing the volatile chemical to evaporate by thermal diffusion, wherein said emission means comprises a ceramic material; and
(c) a connector adapted and arranged to connect said emission means to said light bulb and to position said emission means adjacent to and spaced from said casing.

57. The vapor delivering device of claim 56, wherein said ceramic material includes at least one material selected from a group of materials consisting of a sintered ceramic material and a sintered glass material.

58. The vapor delivering device of claim 56, wherein said ceramic material comprises grains having rounded angles.

59. The vapor delivering device of claim 56, wherein said ceramic material comprises substantially homogeneous grains, and wherein said grains have a size between about 40 microns and about 400 microns.

60. The vapor delivering device of claim 59, wherein the size of said grains is in at least one range selected from a group of ranges consisting of from about 200 microns to about 400 microns, and from about 40 microns to about 80 microns.

61. The vapor delivering device of claim 56, wherein said ceramic material comprises a substantially mono-size pore distribution and wherein a range of pore sizes is in at least one range selected from a group of ranges consisting of from about 0.2 microns to about 110 microns, from about 10 microns to about 20 microns, from about 20 microns to about 30 microns, and from about 7 microns to about 20 microns.

62. The vapor delivering device of claim 56, wherein an overall porosity of the ceramic material is from about 10% to about 80%.

63. The vapor delivering device of claim 56, wherein an overall porosity of the ceramic material is from about 30% to about 50%.

64. The vapor delivering device of claim 56, wherein said ceramic material comprises at least one element selected from a group of elements consisting of a ceramic disc, and a ceramic elongate projection.

65. The vapor delivering device of claim 56, wherein said ceramic material comprises a ceramic rod.

66. The vapor delivering device of claim 65, wherein said connector comprises a retainer formed of a resilient deformable material, said retainer being constructed and arranged to connect to said casing by a push fit, and wherein said retainer further comprises an aperture sized to retain said ceramic rod.

67. The vapor delivering device of claim 66, wherein said emission means is in fluid communication with a reservoir, and wherein said reservoir is coupled to said connector such that said reservoir is remote from said ceramic rod while the vapor delivering device is in use.

68. The vapor delivering device of claim 56, further comprising a detachable envelope adapted to attach to said light fitting and provided with apertures arranged to allow diffusion of the vapor.

69. The vapor delivering device of claim 56, wherein said volatile chemical is at least one volatile chemical selected from a group of volatile chemicals consisting of pyrethrum and a fragrant oil.

70. A vapor delivering device for the evaporation of a volatile chemical into the atmosphere by thermal diffusion comprising:
(a) emission means,
(b) a thermal source; and
(c) a connector, wherein said connector is adapted to secure said emission means to or in the vicinity of said thermal source and wherein the emission means comprises a ceramic material.

71. The vapor delivering device of claim 70, wherein said volatile chemical is at least one volatile chemical selected from a group of volatile chemicals consisting of pyrethrum and a fragrant oil.
